(19) United States
(12) Patent Application Publication
(10) Pub. No.: US 2024/0032993 A1
(43) Pub. Date: Feb. 1, 2024

(54) ELECTRIC WIRE CONNECTION STRUCTURE, ELECTRIC WIRE CONNECTION METHOD, MEDICAL DEVICE, AND METHOD OF MANUFACTURING MEDICAL DEVICE

(71) Applicant: Proterial, Ltd., Tokyo (JP)

(72) Inventor: Ryuta TAKAHASHI, Tokyo (JP)

(21) Appl. No.: 18/217,075

(22) Filed: Jun. 30, 2023

(30) Foreign Application Priority Data
Jul. 26, 2022 (JP) 2022-118563

(51) Int. Cl. A61B 18/14

(52) U.S. Cl. CPC A61B 18/1492; A61B 2018/00083

(57) ABSTRACT

An electric wire connection structure is provided with a plurality of insulated electric wires with each core wire being covered with an insulation coating. The core wires are connected to pads provided on a substrate. The plurality of insulated electric wires are arranged parallel to each other on the substrate along a predetermined alignment direction. The insulation coatings are removed in part in respective longitudinal directions to expose the core wires, and exposed portions of the core wires are connected to the pads, respectively. The exposed portions of the core wires are arranged along an inclined direction inclined with respect to a longitudinal direction of the plurality of insulated electric wires and the alignment direction.

### CROSS-REFERENCE TO RELATED APPLICATIONS

The present patent application claims the priority of Japanese patent application No. 2022-118563 filed on Jul. 26, 2022, and the entire contents thereof are hereby incorporated by reference.

### TECHNICAL FIELD

This invention relates to an electric wire connection structure, an electric wire connection method, a medical device, and a method for manufacturing a medical device.

### BACKGROUND OF THE INVENTION

Conventionally, among medical catheter cables that are configured to be inserted into the human body for examination and treatment, there are those in which a plurality of electric wires are collectively housed in a tubular jacket. A plurality of electrical wires are led out of the jacket at the end of the catheter cable and connected, for example, to a substrate. Patent Literatures 1 and 2 propose structures for connecting a plurality of electric wires and a substrate (i.e., board).

Patent Literature 1 describes a multi-layered structure in which a plurality of coaxial lines are divided into a plurality of groups stacked in a thickness direction of a substrate, and center conductors of respective groups of coaxial lines are connected to contact conductors provided on the surface of the substrate, respectively. The length of the coaxial line varies from group to group, and the center conductor is connected to the contact conductor of the substrate at each tip of each coaxial line.

Patent Literature 2 discloses that a portion of a core wire from which an outer covering (i.e., sheath) made of an insulating material has been removed is inserted into a metal tube, and a connection land of the substrate and the metal tube as well as the core wire are joined by diffusion bonding or ultrasonic bonding while pressing the metal tube from above, in order to improve workability when connecting the core wire made of stranded wire to the connection land of the substrate.

Citation List Patent Literature 1: JP2011-82042A

Patent Literature 2: WO2018/105391A1

### SUMMARY OF THE INVENTION

Medical catheter cables are designed to have a small diameter in order to reduce

the burden on the human body. For example, ultra-thin (i.e., superfine) electric wires corresponding to AWG46 (according to the American Wire Gauge standard) are used as described in Patent Literature 1. However, as the electric wires become thinner, the difficulty of connecting them to the substrate increases.

Therefore, it is an object of the present invention to provide an electric wire connection structure, an electric wire connection method, a medical device, and a method for manufacturing a medical device that enables easy connection of a plurality of electric wires and a substrate.

For solving the above problem, one aspect of the present invention provides: an electric wire connection structure, comprising: a plurality of insulated electric wires with each core wire being covered with an insulation coating, the core wires being connected to pads provided on a substrate, wherein the plurality of insulated electric wires are arranged parallel to each other on the substrate along a predetermined alignment direction, wherein the insulation coatings are removed in part in respective longitudinal directions to expose the core wires, exposed portions of the core wires are connected to the pads, respectively, and the exposed portions of the core wires are arranged along an inclined direction inclined with respect to a longitudinal direction of the plurality of insulated electric wires and the alignment direction.

For solving the above problem, another aspect of the present invention provides: An electric wire connection method for connecting respective core wires of a plurality of insulated electric wires with each core wire being covered with an insulation coating to a pad provided on a substrate, the method comprising: an alignment step of arranging the plurality of insulated electric wires parallel to each other along an alignment direction; an insulation coating removal step of removing the insulation coating of each of the plurality of insulated electric wires in a part in a longitudinal direction to expose the core wire; and a connection step of arranging the plurality of insulated electric wires on the substrate and connecting the core wire of each of the plurality of insulated electric wires to the pad, wherein the insulation coating is removed to expose the core wire at a position along an inclined direction inclined with respect to the longitudinal direction of the plurality of insulated electric wires and the alignment direction.

For solving the above problem, a still another aspect of the present invention provides: a medical device, comprising: a catheter cable comprising the plurality of insulated electric wires with the core wires each covered with the insulation coating; and the substrate having the plurality of pads to which the core wires of the plurality of insulated electric wires are connected, wherein the one end of the catheter cable in the longitudinal direction is configured to be inserted into the human body, wherein the respective core wires of the plurality of insulated electric wires and the plurality of pads of the substrate are connected by the electric wire connection structure as described above.

For solving the above problem, a further aspect of the present invention provides: a method for manufacturing a medical device including a catheter cable having the plurality of insulated electric wires with the core wires each covered with the insulation coating, and the substrate having the plurality of pads to which the core wires of the plurality of insulated electric wires are connected, wherein the one end of the catheter cable in the longitudinal direction is configured to be inserted into the human body, the method comprising: connecting the respective core wires of the plurality of insulated electric wires and the plurality of and the pad by the electric wire connection method as described above.

### Effects of the Invention

According to the present invention, it is possible to provide an electric wire connection structure, an electric wire connection method, a medical device, and a method for manufacturing a medical device that enables easy connection of a plurality of electric wires and a substrate.

### BRIEF DESCRIPTION OF DRAWINGS

FIG. 1A is an explanatory diagram of a multi-electrode catheter in use as an example of a medical device according to an embodiment of the present invention.

FIG. 1B is a cross-sectional view of a catheter cable.

FIGS. 2A to 2C are configuration diagrams showing a multicore wire array in which a plurality of insulated electric wires are arranged in a line along a predetermined alignment direction and integrated.

FIGS. 3A and 3B are plan views showing a substrate provided with a plurality of pads to which respective core wires of the plurality of insulated electric wires forming the multicore wire array are connected.

FIG. 3C is a configuration diagram showing an end surface in a longitudinal direction of the substrate.

FIG. 4A is a side view showing a wire connection body in which the plurality of insulated electric wires and the substrate are combined.

FIG. 4B is an A-A line cross-sectional view of FIG. 4A.

FIG. 5 is a perspective view showing the plurality of insulated electric wires in an insulation coating removal step.

FIG. 6 is an explanatory diagram showing a connection step.

FIG. 7 is an explanatory diagram showing a modified example of the connection step.

FIGS. 8A and 8B are explanatory diagrams showing a modified example in which the plurality of insulated wires are integrated using a belt-like member.

FIG. 9A is an explanatory diagram showing a modified example of positions of exposed portions in the longitudinal direction of the plurality of insulated electric wires.

FIG. 9B is a plan view showing one surface of the substrate combined with the plurality of insulated electric wires shown in FIG. 9A.

FIG. 10A is an explanatory diagram showing another modified example of the positions of the exposed portions in the longitudinal direction of the plurality of insulated electric wires.

FIG. 10B is a plan view showing one surface of the substrate combined with the plurality of insulated electric wires shown in FIG. 10A.

### DETAILED DESCRIPTION OF THE INVENTION

Embodiment

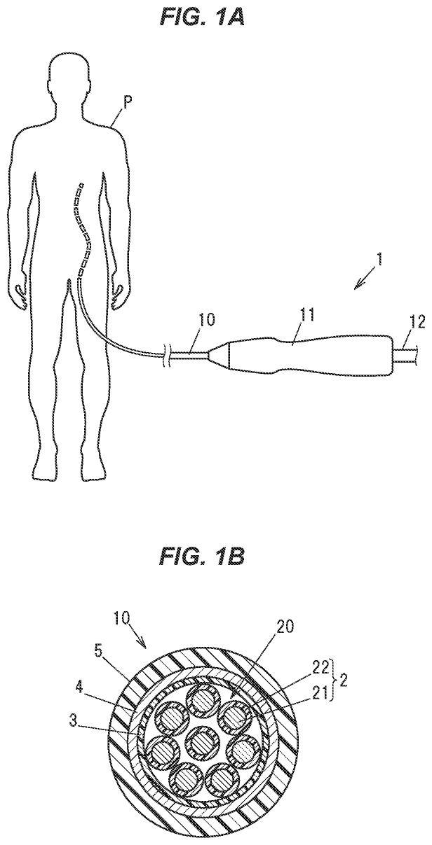

FIG. 1A is an explanatory diagram of a multi-electrode catheter 1 in use as an example of a medical device according to an embodiment of the present invention. FIG. 1B is a cross-sectional view of a catheter cable 10.

The multi-electrode catheter 1 has the catheter cable 10 and a handle 11 to be operated by an operator such as a physician. One longitudinal end portion of the catheter cable 10 is held within the handle 11, and the other longitudinal end portion is inserted into the human body of the subject P for examination or treatment. In FIG. 1A, the catheter cable 10 is shown as a dashed line in the portion inserted within the human body of the subject P.

As shown in FIG. 1B, the catheter cable 10 has an electric wire bundle 20 composed of a plurality of insulated electric wires (i.e., insulated wires) 2, a binder tape 3 wrapped around the outer periphery of the electric wire bundle 20, a shield conductor 4 disposed around the outer periphery of the binder tape 3 and a tubular jacket 5 as an outer covering disposed around the outer periphery of the shield conductor 4. The jacket 5 is made of, e.g., fluoropolymer resin, and houses the electric wire bundle 20, the binder tape 3, and the shield conductor 4. The electric wire bundle 20 is composed of eight insulated electric wires 2 bundled together in this embodiment. Fibrous or string-like fillers (i.e., inclusions) may be placed between the plurality of insulated electric wires 2 in the inner side of the binder tape 3.

Each insulated electric wire 2 has a core wire 21 made of a good electrically conductive metal such as copper and an insulation coating 22 covering the outer periphery of the core wire 21. In the present embodiment, the insulated electric wires 2 are enameled wires, and the core wire 21, which is made of a solid wire of circular cross-section, is covered with an insulation coating 22 made of a resin composition such as polyurethane. The diameter of the core wire 21 is, e.g., 0.02 mm or more and 0.10 mm or less. In the present embodiment, the core wire 21 corresponds to AWG48 according to the AWG (American Wire Gauge) standard, and the conductor diameter is 0.032 mm (32 μm). The thickness of the insulation coating 22 is, e.g., 0.008 mm (8 μm).

The plurality of insulated electric wires 2 are led out of the jacket 5 in the handle 11. A console cable 12 leads from the handle 11, which connects the handle 11 to an unshown console. The console is an information processing device equipped with a microprocessor, memory, etc., which amplifies signals sent from the human body of the subject P via, e.g., a plurality of insulated electric wires 2, and outputs image signals for displaying the state in the inside of the body of the subject P obtained by the amplified signals.

The handle 11 houses a substrate having a plurality of pads to which the core wire 21 of the plurality of insulated electric wires 2 are connected. Signals sent from the human body of the subject P are relayed by this substrate and sent to the console by the console cable 12. Next, the electric wire connection structure in which the core wires 21 of the plurality of insulated electric wires 2 are connected to the plurality of pads on the substrate will be described.

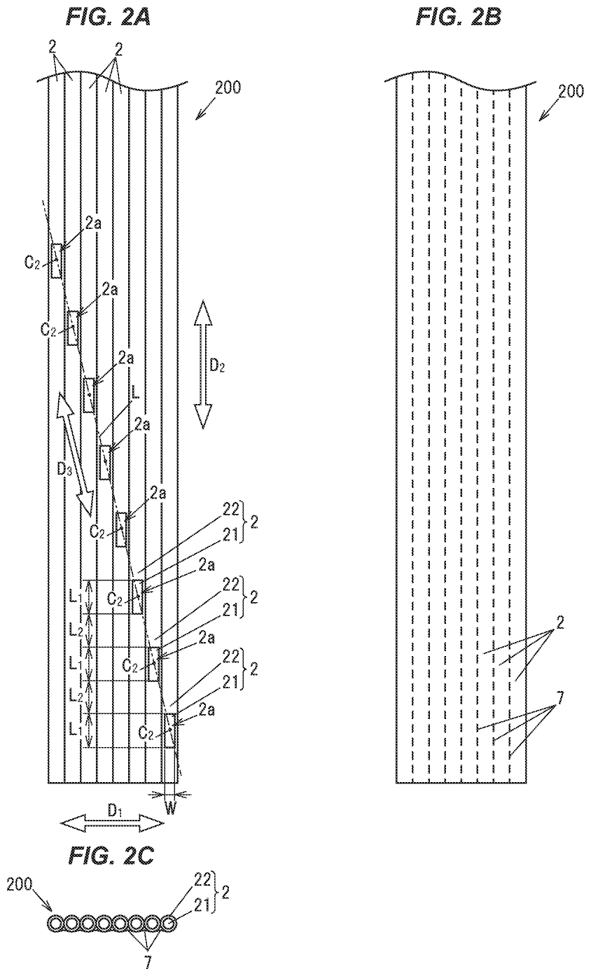

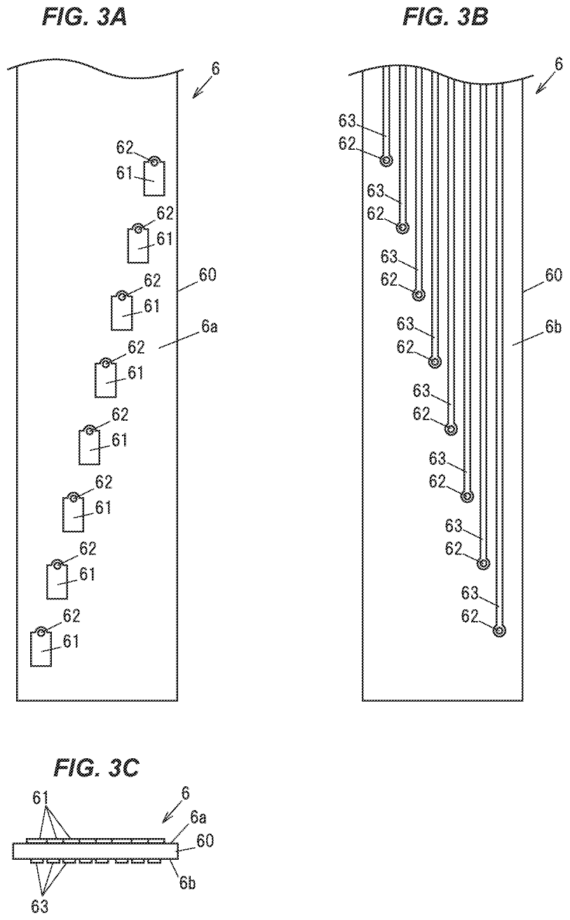

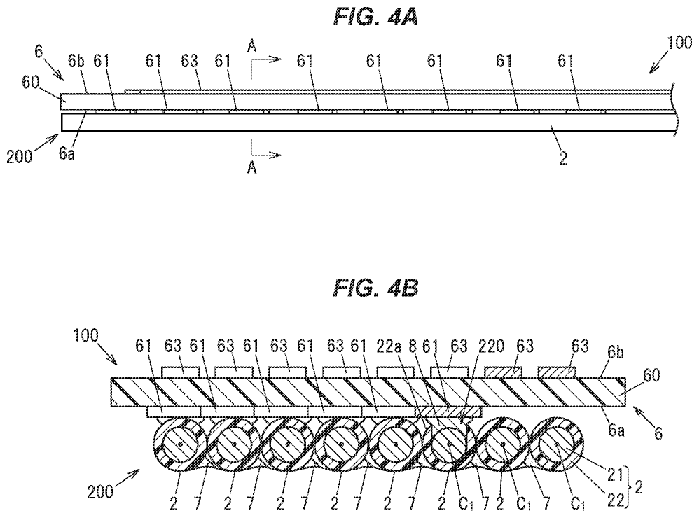

FIGS. 2A to 2C are configuration diagrams showing a multicore wire array 200 in which the plurality of insulated electric wires 2 are arranged in a line along a predetermined alignment direction and integrated. FIGS. 3A and 3B are plan views showing a substrate 6 provided with a plurality of pads 61 to which respective core wires 21 of the plurality of insulated electric wires 2 forming the multicore wire array 200 are connected. FIG. 3C is a configuration diagram showing an end surface in a longitudinal direction of the substrate 6. FIG. 4A is a side view showing a wire connection body 100 in which the plurality of insulated electric wires 2 and the substrate 6 are combined. FIG. 4B is an A-A line cross-sectional view of FIG. 4A.

The plurality of insulated electric wires 2 are integrated at least on the substrate 6 to the multicore wire array 200. The term “on the substrate 6” here refers to a position in close proximity to and facing the substrate 6. FIG. 2A shows a surface of the multicore wire array 200 on a substrate 6-side, and FIG. 2B shows an opposite surface thereof. FIG. 2C shows a state in which the end surfaces of the plurality of insulated electric wires 2 are viewed along the longitudinal direction. In the present embodiment, the plurality of insulated electric wires 2 are integrated by bonding the insulation coatings 22 of the plurality of insulated electric wires 2 with an adhesive 7. As shown in FIG. 4B, the adhesive 7 is applied to the side farther from the substrate 6 than a center line C₁ of each of the core wires 21 of the plurality of insulated electric wires 2, so that no adhesive 7 is interposed between the plurality of insulated electric wires 2 and the substrate 6.

The plurality of insulated electric wires 2 are aligned in a predetermined alignment direction and arranged parallel to each other on the substrate 6, and the insulation coating 22 is removed at a part of each in the longitudinal direction to expose the core wire 21. This exposed portion of the core wire 21 is connected to the pad 61. The direction in which the plurality of insulated electric wires 2 are arranged is parallel to the substrate 6 and orthogonal to the longitudinal direction of each insulated electric wire 2. In FIG. 2A, the alignment direction D₁ and the longitudinal direction D₂ of the plurality of insulated electric wires 2 are indicated by arrows, respectively.

The insulation coating 22 of each of the plurality of insulated electric wires 2 is removed from a portion of an outer periphery of the core wire 21 including a portion between the core wire 21 and the pad 61, and the surface of the core wire 21 opposite to the pad 61 is covered with an insulation coating 22. More specifically, as shown in FIG. 4B, a portion of the insulation coating 22 is removed so that the insulation coating 22 has a C-like cross-section, and a recess 220 that is recessed from an outer peripheral surface 22a of the insulation coating 22 toward the core wire 21 is formed. The core wire 21 and the pad 61 are electrically connected by a solder 8 accommodated in the recess 220.

The respective exposed portions of the core wires 21 of the plurality of insulated electric wires 2 are arranged in a direction inclined with respect to the alignment direction D₁ and the longitudinal direction D₂ of the plurality of insulated electric wires 2. Hereinafter, the portion where the core wire 21 is exposed is referred to as an exposed portion 2a. In FIG. 2A, a straight line L connecting the center points C₂ of the exposed portions 2a is indicated by a dashed line, and an inclined direction D₃ indicating the direction of this straight line L is indicated by an arrow.

As shown in FIG. 2A, the exposed portions 2a of a pair of adjacent insulated electric wires 2 are not aligned in the alignment direction D₁. Therefore, of the eight insulated electric wires 2, the exposed portions 2a of the six insulated electric wires 2 excluding the two insulated electric wires 2 at both ends in the alignment direction D₁ are sandwiched by the insulation coatings 22 of the adjacent insulated electric wires 2. This configuration prevents the core wires 21 of the plurality of insulated electric wires 2 from being bridge-connected by the solder 8. The width W of the exposed portion 2a in the alignment direction D₁ is equivalent to the diameter of the core wire 21.

When the length of the exposed portion 2a in the longitudinal direction D₂ is L₁, and an interval (i.e., distance) between the exposed portions 2a of the plurality of insulated electric wires 2 in the longitudinal direction D₂ is L₂, L₂ is shorter than L₁ and longer than the outer diameter of the insulated electric wire 2. As a result, the length of the substrate 6 in the longitudinal direction D₂ is suppressed from becoming unnecessarily long while suppressing the occurrence of solder bridges. Note that L₂ does not necessarily have to be the same at all sites, and may be non-uniform.

In this embodiment, as an example, eight insulated electric wires 2 are arranged in parallel to form the multicore wire array 200, but the number of insulated electric wires 2 is not limited, and the number may be three or more. That is, it is sufficient that the portions where the core wires 21 of at least three insulated electric wires 2 are exposed are arranged in a direction inclined with respect to the longitudinal direction and the alignment direction of these insulated electric wires 2.

The substrate 6 is an FPC (flexible printed circuit board), and the plurality of pads 61 are provided on the surface of a flat substrate member (i.e., base material) 60. The substrate member 60 is made of an insulating material, such as polyimide, and is flexible. FIG. 3A shows one surface (first surface) 6a of the substrate member 60, and FIG. 3B shows the other surface (second surface) 6b, which is the back side of the one surface 6a. FIG. 3C shows a longitudinal end surface of the substrate 6.

The substrate 6 is provided with a through-hole 62 that penetrates the substrate member 60 in the thickness direction and a linear wiring pattern 63. In the example shown in FIGS. 3A and 3B, the through-hole 62 is provided in a part of each of the plurality of pads 61, and the wiring pattern 63 extending from the through-hole 62 is provided on the other surface 6b-side. The insulated electric wire 2 contacts the one surface 6a of the substrate member 60 over a wide range. Through-holes 62 may be provided in the vicinity of the plurality of pads 61 via lead wires. By forming the wiring pattern 63 mainly on the side of the other surface 6b, the insulated electric wires 2 face the one surface 6a of the flat substrate member 60 over a wide range, thereby facilitating the alignment (i.e., positioning) between the multicore wire array 200 and the substrate 6 with high accuracy.

The pads 61 and the wiring patterns 63 are metal foils formed by etching, and their thickness is, e.g., 1 μm or more and 16 μm or less. The core wire 21 of the insulated electric wire 2 is electrically connected to the terminals of an unshown electronic component mounted on the substrate 6, e.g., via the pad 61, the through-hole 62, and the wiring pattern 63.

Next, a wire connection method for connecting the respective core wires 21 of the plurality of insulated electric wires 2 to the pads 61 provided on the substrate 6 will be described. This wire connection method includes an alignment step of arranging a plurality of insulated electric wires 2 parallel to each other along the alignment direction D₁, an integration step of integrating the plurality of insulated electric wires 2 arranged in the alignment step, an insulation coating removal step of removing the insulation coating 22 of each of the plurality of insulated electric wires 2 in a part in the longitudinal direction D₂ to expose the core wire 21 in the state where the plurality of insulated electric wires 2 are integrated, and a connection step of arranging the plurality of insulated electric wires 2, from which the insulation coatings 22 have been removed in part, on the substrate 6 and connecting the respective core wires 21 of the insulated electric wires 2 to the pads 61. The multi-electrode catheter 1 is manufactured by a manufacturing method using this wire connection method.

In the alignment step, tension is applied to the plurality of insulated electric wires 2 in the longitudinal direction, and the plurality of insulated electric wires 2 are aligned in parallel with each other in a straight state. In the integration step, the adhesive 7 is applied to the surfaces of the plurality of insulated electric wires 2 opposite to the facing surface of the substrate 6 to integrate the plurality of insulated electric wires 2 to form the multicore wire array 200. In the insulation coating removal step, the insulation coating 22 of each of the plurality of insulated electric wires 2 is removed at a position along the inclined direction D₃ to expose the core wire 21.

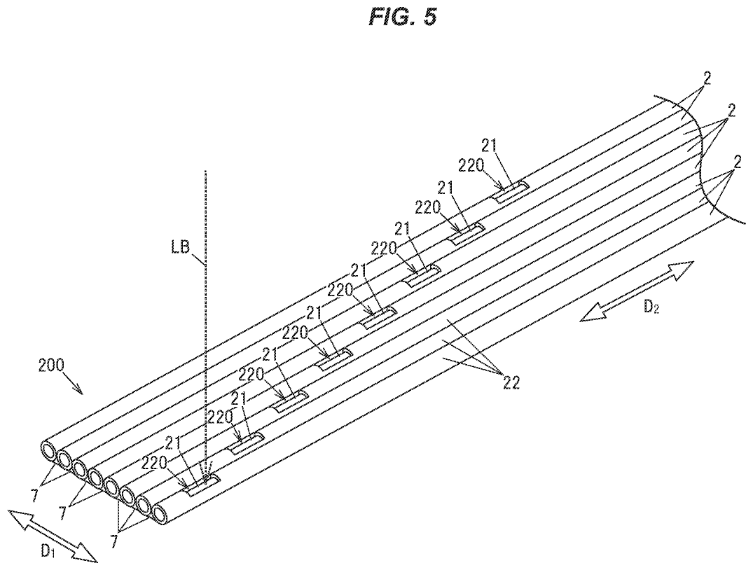

FIG. 5 is a perspective view showing a plurality of insulated electric wires 2 in the insulation coating removal step. In the insulation coating removal step, the insulation coating 22 is partly removed to expose the core wire 21 by irradiating the laser beam LB perpendicularly to the alignment direction D₁ and the longitudinal direction D₂. The beam diameter of the laser beam LB at the portion that irradiates the insulation coating 22 is equivalent to the diameter of the core wire 21, for example. By performing the insulation coating removal step using the laser beam LB, the insulation coating 22 on a part of the outer periphery of the core wire 21 including the portion between the core wire 21 and the pad 61 can be removed just enough. A portion from which the insulation coating 22 is removed becomes the recess 220 that opens toward the substrate 6.

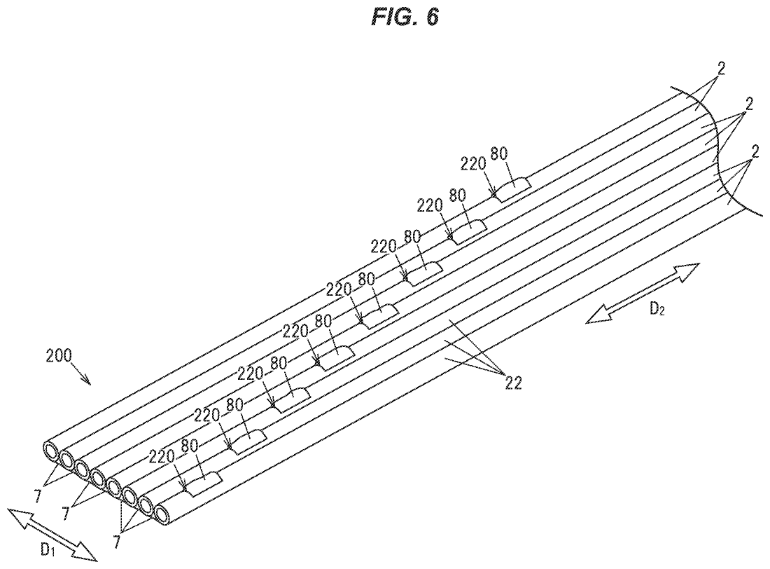

FIG. 6 is an explanatory diagram showing the connection step. In the connection step, as shown in FIG. 6, after applying cream solder 80, which is paste-like solder, to the recesses 220, the one surface 6aof the substrate 6 is brought close to the multicore wire array 200, and the cream solder 80 is applied to the pads 61. After that, the cream solder 80 is melted by heating, and the core wire 21 is connected to the pad 61 by soldering as shown in FIG. 4B. The heat resistance temperature of the insulation coating 22 is higher than the melting point of the cream solder 80. When the core wire 21 of an insulated electric wire 2 is soldered, even if the molten solder 8 contacts the insulation coating 22 of the other insulated electric wire 2 adjacent to the insulated electric wire 2, the insulation coating 22 does not melt.

Effects of Embodiment

According to the embodiment of the present invention described above, the exposed portions 2a where the core wires 21 are exposed are arranged along the inclined direction D₃ that is inclined with respect to the direction D₁ and the longitudinal direction D₂ of the plurality of insulated electric wires 2. Therefore, the interval between the exposed portions 2a can be increased compared to, for example, the case where the exposed portions 2a are arranged along the alignment direction D₁. It is thus possible to easily connect the plurality of insulated electric wires 2 and the substrate 6 while suppressing the occurrence of the solder bridges.

Further, since the insulation coating 22 is removed from a portion of the outer periphery of the core wire 21 including the portion between the core wire 21 and the pad 61, the work can be performed easily as compared to the case where the insulation coating 22 is removed over the entire outer periphery of the core wire 21. In addition, since the cream solder 80 can be held in the recess 220 formed by removing the insulation coating 22, the connection step can be easily and reliably performed.

Still further, since the plurality of insulated electric wires 2 are integrated, it is possible to prevent the positions of the insulated electric wires 2 from being displaced, and to appropriately maintain the intervals between the exposed portions 2a of the plurality of insulated electric wires 2, thereby suppressing the occurrence of the solder bridges. Moreover, by performing the insulation coating removal step and the connection step in a state where the plurality of insulated electric wires 2 are integrated, these steps can be performed easily and reliably. Furthermore, by integrating the plurality of insulated electric wires 2 with the adhesive 7 applied to the side farther from the substrate 6 than the center lines C₁ of the respective core wires 21 of the plurality of insulated electric wires 2, it is possible to prevent the adhesive 7 from being interference with the work of the insulation coating removal step and the connection step.

Modified Examples

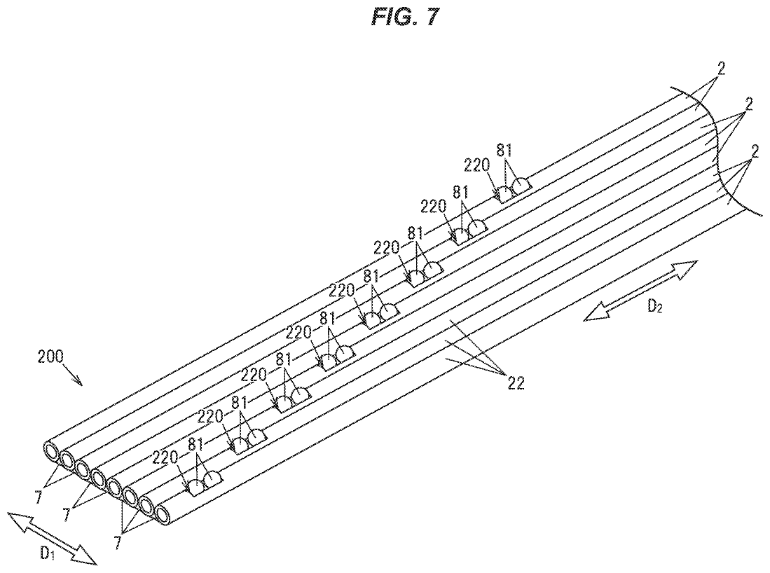

Next, modified examples of the embodiment will be described with reference to FIGS. 7 to 10B. In FIGS. 7 to 10B, the same reference numerals as those in FIGS. 2A to 6 are assigned to the components corresponding to those described in the above embodiment, and redundant description is omitted.

FIG. 7 is an explanatory diagram showing a modified example of the connection step. In the above embodiment, the soldering is performed by applying the cream solder 80 to the recess 220 formed by removing the insulation coating 22. However, in the modified example shown in FIG. 7, for each of the plurality of insulated electric wires 2, a spherical solder ball 81 is accommodated in the recess 220, and the core wire 21 is soldered to the pad 61 by melting the solder ball 81 by heating. Although FIG. 7 illustrates a case where two solder balls 81 are accommodated in each recess 220, the number of solder balls 81 accommodated in each recess 220 is not limited two and may be one, or three or more.

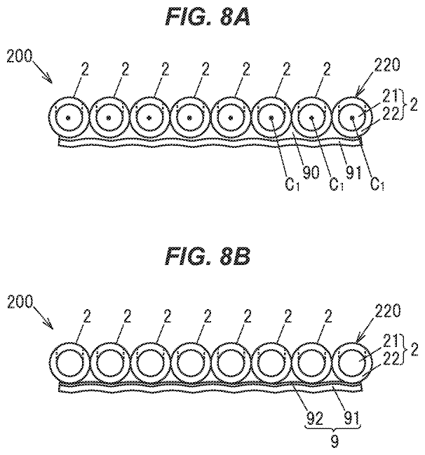

FIGS. 8A and 8B are explanatory diagrams showing a modified example in which the plurality of insulated electric wires 2 are integrated using a belt-like (strip-like) member 91 arranged on the side opposite to the substrate 6. FIG. 8A shows a modified example in which a plurality of insulated electric wires 2 are integrated by bonding the plurality of insulated electric wires 2 to the belt-like member 91 with an adhesive 90. The adhesive 90 is applied to the side farther from the substrate 6 than the center lines C₁ of the respective core wires 21 of the plurality of insulated electric wires 2. In FIG. 8B, an adhesive tape 9 having an adhesive layer 92 formed on one side of the belt-like member 91 is used, and the plurality of insulated electric wires 2 are adhered to the belt-like member 91 by the adhesive layer 92, whereby the plurality of insulated electric wires 2 are integrated. The belt-like member 91 is made of a flexible belt-like resin and is vertically attached so that its longitudinal direction is parallel to the longitudinal direction of the plurality of insulated electric wires 2.

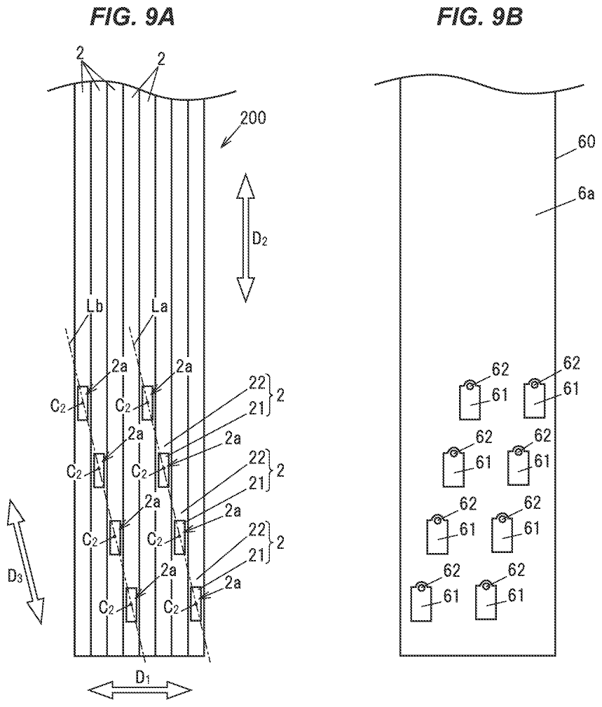

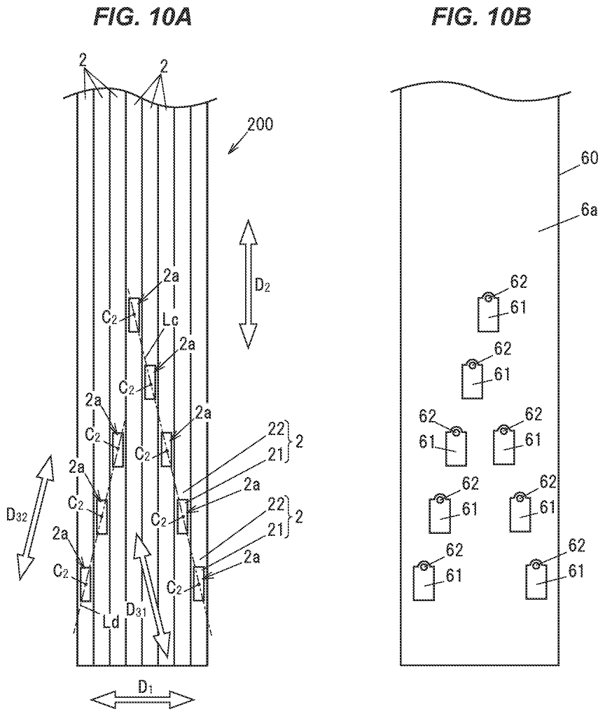

FIGS. 9A and 10A are explanatory diagrams showing modified examples of the positions of the exposed portions 2a in the longitudinal direction of the plurality of insulated electric wires 2. FIG. 9B is a plan view showing the one surface 6a of the substrate 6 combined with the plurality of insulated electric wires 2 shown in FIG. 9A. FIG. 10B is a plan view showing the one surface 6a of the substrate 6 combined with the plurality of insulated electric wires 2 shown in FIG. 10A. In the above embodiment, the case where all the exposed portions 2a of the eight insulated electric wires 2 forming the multicore wire array 200 are arranged along a single straight line L has been described. In the modified examples shown in FIGS. 9A and 10A, the arrangement of the exposed portions 2a is different from that of the above-described embodiment. In the modified example shown in FIG. 9A, the exposed portions 2a of four of the

eight insulated electric wires 2 forming the multicore wire array 200 are aligned along a first straight line La, and the exposed portions 2a of the other four insulated electric wires 2 are arranged along the second straight line Lb. The first straight line La and the second straight line Lb both extend along the inclined direction D₃ that is inclined with respect to the alignment direction D₁ and the longitudinal direction D₂, and are parallel to each other. The exposed portions 2a of the four insulated electric wires 2 aligned along the first straight line La and the exposed portions 2a of the other four insulated electric wires 2 aligned along the second straight line Lb are aligned in the alignment direction D₁ via the three insulated electric wires 2, respectively.

In the modified example shown in FIG. 10A, the exposed portions 2a of five of the eight insulated electric wires 2 forming the multicore wire array 200 are aligned along a third straight line Lc. The exposed portions 2a of the other three of the eight insulated electric wires 2 are arranged along a fourth straight line Ld. The third straight line Lc and the fourth straight line Ld are both inclined with respect to the alignment direction D₁ and the longitudinal direction D₂, but the directions of inclination with respect to the longitudinal direction D₂ are opposite to each other. The extending direction of the third straight line Lc is an inclined direction D₃₁ which is inclined to one side with respect to the longitudinal direction D₂, and the extending direction of the fourth straight line Ld is an inclined direction D₃₂ which is inclined to the other side with respect to the longitudinal direction D₂. The exposed portions 2a of the three insulated electric wires 2 aligned along the fourth straight line Ld and the exposed portions 2a of three of the five insulated electric wires 2 aligned along the third straight line Lc are aligned in the alignment direction D₁ via a plurality of insulated electric wires 2, respectively. Each of these modified examples can also achieve the same effects and effects as

the above-described embodiment. In addition, although The electric wire connection structure at the one ends of the plurality of insulated electric wires 2 is shown in the above embodiment and each modified example, the core wires 21 may be connected to the plurality of pads 61 on the substrate 6 by means of The electric wire connection structure at the other ends in the longitudinal direction of the plurality of insulated electric wires 2, similarly. In this case, by making the arrangement of the exposed portions 2a of the multicore wire array 200 at one end of the plurality of insulated electric wires 2 the same as the arrangement of the exposed portions 2a of the multicore wire array 200 at the other end, the distance between the connecting portion between the core wire 21 and the pad 61 of the substrate 6 at one end of each insulated electric wire 2 and the connecting portion between the core wire 21 and the pad 61 of the substrate 6 at the other end can be made common.

Summary of Embodiment and Modified Examples

Next, technical ideas understood from the embodiment and modified examples described above will be described with reference to the reference numerals and the like in the embodiment and modified examples. However, each reference numeral in the following description does not limit the constituent elements in the claims to the members and the like specifically shown in the embodiment.

According to the first feature, in an electric wire connection structure, in which respective core wires 21 of a plurality of insulated electric wires 2 with each core wire 21 being covered with an insulation coating 22 are connected to pads 61 provided on a substrate 6, the plurality of insulated electric wires 2 are arranged parallel to each other on the substrate 6 along a predetermined alignment direction D₁, and the insulation coatings 22 are removed in part in respective longitudinal directions to expose the core wires 21, the exposed portions 2a of the core wires 21 are connected to the pads 61, respectively, and the exposed portions 2a of the core wires 21 are arranged along an inclined direction D₃, D₃₁, D₃₂ that is inclined with respect to a longitudinal direction D₂ of the plurality of insulated electric wires 2 and the alignment direction D₁.

According to the second feature, in The electric wire connection structure as described in the first feature, the insulation coating 22 is removed at a portion of an outer periphery of the core wire 21 including a portion between the core wire 21 and the pad 61, and, for the core wire 21, a surface opposite to the pad 61 is covered with the insulation coating 22.

According to the third feature, in the electric wire connection structure as described in the first feature, the exposed portions 2a of the core wires 21 of at least three of the plurality of insulated electric wires 2 are arranged along the inclined direction D₃, D₃₁, D₃₂.

According to the fourth feature, in The electric wire connection structure as described in the first feature, the plurality of insulated electric wires 2 are integrated at least on the substrate 6.

According to the fifth feature, in the electric wire connection structure as described in the fourth feature, the plurality of insulated electric wires 2 are integrated by bonding the insulation coatings 22 of the plurality of insulated electric wires 2 with an adhesive 7.

According to the sixth feature, in the electric wire connection structure as described in the fifth feature, the adhesive 7 is applied on a side farther from the substrate 6 than a center line C₁ of the core wire 21 of each of the plurality of insulated electric wires 2.

According to the seventh feature, in the electric wire connection structure as described in the fourth feature, the plurality of insulated electric wires 2 are integrated by adhesion or sticking to a belt-like member 91 arranged on a side opposite to the substrate 6.

According to the eighth feature, in the electric wire connection structure as described in the fifth feature, the core wire 21 has a diameter of 0.1 mm or less.

According to the ninth feature, an electric wire connection method for connecting respective core wires 21 of a plurality of insulated electric wires 2 with each core wire 21 being covered with an insulation coating 22 to a pad 61 provided on a substrate 6, the method comprising: an alignment step of arranging the plurality of insulated electric wires 2 parallel to each other along an alignment direction D₁, an insulation coating removal step of removing the insulation coating 22 of each of the plurality of insulated electric wires 2 in a part in a longitudinal direction D₂ to expose the core wire 21, and a connection step of arranging the plurality of insulated electric wires 2 on the substrate 6 and connecting the core wire 21 of each of the plurality of insulated electric wires 2 to the pad 61, wherein the insulation coating 22 is removed to expose the core wire 21 at a position along an inclined direction D₃, D₃₁, D₃₂ that is inclined with respect to the longitudinal direction D₂ of the plurality of insulated electric wires 2 and the alignment direction D₁.

According to the tenth feature, the electric wire connection method as described in the ninth feature further includes an integration step of integrating the plurality of insulated electric wires 2 arranged in the alignment step, wherein, in the insulation coating removal step, the insulation coating 22 is removed in the state where the plurality of insulated electric wires 2 are integrated.

According to the eleventh feature, in the electric wire connection method as described in the ninth feature, in the insulation coating removing step, the insulation coating 22 is removed at a portion of an outer periphery of the core wire 21 including a portion between the core wire 21 and the pad 61.

According to the twelfth feature, in the electric wire connection method as described in the eleventh feature, in the connection step, the core wire 21 is soldered to the pad 61 by melting a paste-like solder 80 adhered to a portion from which the insulation coating 22 has been removed in the insulation coating removal step.

According to the thirteenth feature, in the electric wire connection method as described in the eleventh feature, in the connection step, the core wire 21 is soldered to the pad 61 by melting a solder ball 81 accommodated in a portion from which the insulation coating 22 has been removed in the insulation coating removal step.

According to the fourteenth feature, a medical device (multi-electrode catheter) 1 includes a catheter cable 10 having a plurality of insulated electric wires 2 with core wires 21 each covered with an insulation coating 22, and a substrate 6 having a plurality of pads 61 to which the core wires 21 of the plurality of insulated electric wires 2 are connected, wherein one end of the catheter cable 10 in a longitudinal direction is configured to be inserted into a human body, wherein the respective core wires 21 of the plurality of insulated electric wires 2 and the plurality of pads 61 of the substrate 6 are connected by the electric wire connection structure as described in one of the first feature to the eighth feature.

According to the fifteenth feature, a method for manufacturing a medical device 1 including a catheter cable 10 having a plurality of insulated electric wires 2 with core wires 21 each covered with an insulation coating 22, and a substrate 6 having a plurality of pads 61 to which the core wires 21 of the plurality of insulated electric wires 2 are connected, wherein one end of the catheter cable 10 in a longitudinal direction is configured to be inserted into a human body, the method includes: connecting the respective core wires 21 of the plurality of insulated electric wires 2 and the plurality of and the pad 61 by the electric wire connection method according to any one of the ninth feature to the thirteenth feature.

Although the embodiment and modified examples of the present invention have been described above, the embodiment and modified examples described above do not limit the scope of the invention according to the claims. Also, it should be noted that not all combinations of features described in the embodiments are essential to the means for solving the problems of the invention.

Further, in the above embodiment, the case where the present invention is applied to the multi-electrode catheter 1, which is a kind of medical device, has been described, but the present invention is not limited to this, and for example, the present invention can be applied to an endoscope. Alternatively, the present invention may be applied to devices other than medical devices. Further, in the above-described embodiment, the case where the core wire 21 is connected to the pad 61 by soldering has been described, but the invention is not limited to this, and the core wire 21 may be connected to the pad 61 with an electrically conductive adhesive.

1. An electric wire connection structure, comprising:
a plurality of insulated electric wires with each core wire being covered with an insulation coating, the core wires being connected to pads provided on a substrate, wherein the plurality of insulated electric wires are arranged parallel to each other on the substrate along a predetermined alignment direction, wherein the insulation coatings are removed in part in respective longitudinal directions to expose the core wires, exposed portions of the core wires are connected to the pads, respectively, and the exposed portions of the core wires are arranged along an inclined direction inclined with respect to a longitudinal direction of the plurality of insulated electric wires and the alignment direction.

2. The electric wire connection structure, according to claim 1, wherein the insulation coating is removed at a portion of an outer periphery of the core wire including a portion between the core wire and the pad, and, for the core wire, a surface opposite to the pad is covered with the insulation coating.

3. The electric wire connection structure, according to claim 1, wherein the exposed portions of the core wires of at least three of the plurality of insulated electric wires are arranged along the inclined direction.

4. The electric wire connection structure, according to claim 1, wherein the plurality of insulated electric wires are integrated at least on the substrate.

5. The electric wire connection structure, according to claim 4, wherein the plurality of insulated electric wires are integrated by bonding the insulation coatings of the plurality of insulated electric wires with an adhesive.

6. The electric wire connection structure, according to claim 5, wherein the adhesive is applied on a side farther from the substrate than a center line of the core wire of each of the plurality of insulated electric wires.

7. The electric wire connection structure, according to claim 4, wherein the plurality of insulated electric wires are integrated by adhesion or sticking to a belt-like member arranged on a side opposite to the substrate.

8. The electric wire connection structure, according to claim 5, wherein the core wire has a diameter of 0.1 mm or less.

9. An electric wire connection method for connecting respective core wires of a plurality of insulated electric wires with each core wire being covered with an insulation coating to a pad provided on a substrate, the method comprising:
an alignment step of arranging the plurality of insulated electric wires parallel to each other along an alignment direction;
an insulation coating removal step of removing the insulation coating of each of the plurality of insulated electric wires in a part in a longitudinal direction to expose the core wire; and
a connection step of arranging the plurality of insulated electric wires on the substrate and connecting the core wire of each of the plurality of insulated electric wires to the pad,
wherein the insulation coating is removed to expose the core wire at a position along an inclined direction inclined with respect to the longitudinal direction of the plurality of insulated electric wires and the alignment direction.

10. The electric wire connection method, according to claim 9, further comprising:
an integration step of integrating the plurality of insulated electric wires arranged in the alignment step,
wherein, in the insulation coating removal step, the insulation coating is removed in the state where the plurality of insulated electric wires are integrated.

11. The electric wire connection method, according to claim 9, wherein, in the insulation coating removing step, the insulation coating is removed at a portion of an outer periphery of the core wire including a portion between the core wire and the pad.

12. The electric wire connection method, according to claim 11, wherein, in the connection step, the core wire is soldered to the pad by melting a paste-like solder adhered to a portion from which the insulation coating has been removed in the insulation coating removal step.

13. The electric wire connection method, according to claim 11, wherein, in the connection step, the core wire is soldered to the pad by melting a solder ball accommodated in a portion from which the insulation coating has been removed in the insulation coating removal step.

14. A medical device, comprising:
a catheter cable comprising the plurality of insulated electric wires with the core wires each covered with the insulation coating; and
the substrate having the plurality of pads to which the core wires of the plurality of insulated electric wires are connected,
wherein the one end of the catheter cable in the longitudinal direction is configured to be inserted into the human body,
wherein the respective core wires of the plurality of insulated electric wires and the plurality of pads of the substrate are connected by the electric wire connection structure according to claim 1.

15. A method for manufacturing a medical device including a catheter cable having the plurality of insulated electric wires with the core wires each covered with the insulation coating, and the substrate having the plurality of pads to which the core wires of the plurality of insulated electric wires are connected, wherein the one end of the catheter cable in the longitudinal direction is configured to be inserted into the human body, the method comprising:
connecting the respective core wires of the plurality of insulated electric wires and the plurality of and the pad by the electric wire connection method according to claim 9.
